(19) United States
(12) Patent Application Publication
(10) Pub. No.: US 2025/0136219 A1
(43) Pub. Date: May 1, 2025

(54) STORAGE KIT FOR PERSONAL MOBILITY APPARATUS

(71) Applicants: Hyundai Motor Company, Seoul (KR); Kia Corporation, Seoul (KR); Hyundai Mobis Co., Ltd., Seoul (KR)

(72) Inventors: Dae Young Kwak, Hwaseong-si (KR); Jang Mook Lim, Hwaseong-si (KR); Mun Seok Jeong, Hwaseong-si (KR); Kyu Sang Lee, Yongin-si (KR); Seok Hoon Ko, Yongin-si (KR); Dae Chang Jung, Hwaseong-si (KR)

(21) Appl. No.: 18/931,720

(22) Filed: Oct. 30, 2024

(30) Foreign Application Priority Data
Oct. 30, 2023 (KR) 10-2023-0146790

(51) Int. Cl. B62J 9/21; A61L 2/10; B62J 3/10; B62J 6/029; B62J 6/03; B62J 9/27; B62J 11/24; B62K 3/00

(52) U.S. Cl. CPC B62J 9/21; A61L 2/10; B62J 3/10; B62J 6/029; B62J 6/03; B62J 9/27; B62J 11/24; B62K 3/002; A61L 2202/11; A61L 2202/122; B62K 2202/00

(57) ABSTRACT

A safety device storage kit for a personal mobility apparatus can safely store a user-wearable airbag module of a personal mobility apparatus from the risk of theft and loss, and can improve the safety by indicating the fact that the personal mobility apparatus is running in the form of a light beam and/or sound from a speaker.

### CROSS-REFERENCE TO RELATED APPLICATIONS

The present application claims priority to Korean Patent Application No. 10-2023-0146790, filed on Oct. 30, 2023, the entire contents of which is incorporated herein for all purposes by this reference.

### TECHNICAL FIELD

The present disclosure relates to a safety device storage kit for a personal mobility apparatus.

### BACKGROUND

Recently, as the use of smart mobility for the first mile or the last mile has become active, sharing services of electric kickboards, etc. are expanding.

In addition, regulations on essential devices to ensure the safety of users when using electric kickboards are increasing, and recently, it has become essential to wear safety devices such as safety helmets for the safety of users when using electric kickboards. This request can be said to be appropriate, especially considering that major injuries are concentrated on the head or face in electric kickboard accidents.

To this end, users of personal mobility apparatuses such as electric kickboards either use their own safety helmets or share safety helmets provided by sharing service providers.

However, due to the inconvenience of the user's head being pressed or matted when wearing a safety helmet and the inconvenience of having to always carry a personally owned safety helmet, it is not widely used.

The safety helmets provided by sharing service providers are not only exposed to the outside, but are also used repeatedly by an unspecified number of people, making it difficult to shake off concerns about contamination and hygiene.

In addition, because personal mobility apparatuses run on the power of electric motors, they generate less noise, making it difficult for pedestrians around them to recognize the approach of personal mobility apparatuses, which has caused safety accidents due to unexpected pedestrian movements.

### SUMMARY

The present disclosure relates to a safety device storage kit, and more specifically, to a safety device storage kit for a personal mobility apparatus, which can safely store and protect a user-wearable airbag module of a personal mobility apparatus from the risk of theft and loss, and improve the safety by indicating the fact that the personal mobility apparatus is running in the form of a light beam.

An embodiment of the present disclosure can provide a safety device storage kit for a personal mobility apparatus, which can safely store and protect a user-wearable airbag module of a personal mobility apparatus from the risk of theft and loss, and improve the safety by indicating the fact that the personal mobility apparatus is running in the form of a light beam.

According to an embodiment of the present disclosure, a storage kit for a personal mobility apparatus can include: a housing configured to accommodate a convenience device module, a cover configured to cover an upper part of the housing, and a fastening part configured to couple the housing to a personal mobility apparatus, where the housing includes a power supply for supplying power to the convenience device module.

The housing may further include a light beam generator configured to project image beam on a ground, and the light beam generator can include a LED configured to generate light, a first lens configured to transmit the light, an image film configured to provide an image to be projected using light passing through the first lens, and a second lens, which can be an outermost lens configured to project the light passing through the image film onto the ground outside the housing.

The light beam generator may further include a distance detector configured to measure a distance between the personal mobility apparatus and a pedestrian or obstacle in front of the personal mobility apparatus.

The distance detector may be an infrared transceiver or ultrasonic sensor installed in the light beam generator.

The light beam generator may transmit a warning sound through a speaker when the distance to the pedestrian is less than a preset or selected distance.

The light beam generator may control a color of the image projected on the ground to be changed from a first color to a second color when a running speed of the personal mobility apparatus exceeds a preset or selected reference speed.

The light beam generator may express the color of the image as the first color when the running speed is smaller than the reference speed, and display the color of the image as the second color when the running speed is greater than the reference speed.

The housing may be provided with a plurality of light beam generators, each capable of independently expressing an image, thereby allowing simultaneous projection of a plurality of images on the ground.

The convenience device module may include an airbag module wearable by a user, the housing may include an inner wall configured to support an inner peripheral surface of the airbag module, and a mounting portion formed concavely at a bottom of the housing to allow for a charging module of the airbag module to be inserted, and the inner wall can be provided with a UVC (ultraviolet spectrum light) LED (light emitting diode) for disinfecting the inner peripheral surface of the airbag module.

The power supply may include a charging terminal provided to the mounting portion and configured to supply power for charging a battery provided in the charging module of the airbag module or a smart terminal, and a first wireless charging transmitter can be installed in the mounting portion and including a coil capable of wirelessly supplying power to a first wireless charging receiver of the charging module of the airbag module.

The cover may include a smartphone mounting portion for mounting a smartphone, and the smartphone mounting portion may include a mounting plate slanted to place the smartphone at an angle, and a grip portion configured to surround both lateral sides and a lower surface of the mounting plate.

A second wireless charging transmitter for wirelessly charging the smartphone can be placed on the mounting plate and may be further provided on a bottom surface of the mounting plate.

The housing may include a Bluetooth module configured to enable wireless communication with the smartphone, and a speaker configured to output audio played by the smartphone.

The housing may include a lighting unit and a stabilizer configured to attenuate vibration transmitted to the lighting unit while the personal mobility apparatus is running.

The stabilizer may include a lower fixing member including a lower fixing surface attached to the personal mobility apparatus, at least one support bar mounted between the lighting unit and the lower fixing member and configured to be compressible to guide movement of the lower fixing member toward the lighting unit, and a spring mounted between the lighting unit and the lower fixing member.

The spring may include a coil spring configured to surround the at least one support bar, or a plate spring mounted between the lighting unit and the lower fixing member.

According to various embodiments of the present disclosure, a user-wearable airbag module of a personal mobility apparatus can be safely stored and protected from the risk of theft and loss, and the airbag module can be automatically sterilized after use so that a hygienic use environment can be maintained.

According to various embodiments of the present disclosure, the navigation function can be utilized while driving a personal mobility apparatus by using a smartphone mounting portion provided on the cover of the housing, and the smartphone can be wirelessly charged.

According to various embodiments of the present disclosure, audio can be played through a speaker connected to a smartphone and Bluetooth, which can be used as a navigation speaker and/or as an audio device while using a personal mobility apparatus.

According to various embodiments of the present disclosure, accidents can be prevented by improving the visibility of surrounding pedestrians through an image projected to the ground, and the intuitive recognition of pedestrians can be improved by changing and displaying the color of the image according to the driving speed of the personal mobility apparatus.

According to various embodiments of the present disclosure, safety accidents can be prevented by measuring the distance to pedestrians and then sending out a warning sound to urge caution.

According to various embodiments of the present disclosure, the visibility of lighting and light beams can be improved by minimizing the shaking of lighting due to vibration or shock generated while driving a personal mobility apparatus.

The advantages that can be obtained from an embodiment of the present disclosure are not necessarily limited to those mentioned above, and other advantages and effects not mentioned can be clearly understood by those skilled in the art from the description below.

### BRIEF DESCRIPTION OF THE DRAWINGS

FIG. 1 is an example view of an electric kickboard to which a safety device storage kit for a personal mobility apparatus according to an embodiment of the present disclosure is applied.

FIG. 2 is an internal perspective view of a housing according to an embodiment of the present disclosure.

FIG. 3 is an example view of an airbag module according to an embodiment of the present disclosure.

FIG. 4 is an example view showing a state in which an airbag module is docked in a housing according to an embodiment of the present disclosure.

FIG. 5 is a cross-sectional view of a mounting portion according to an embodiment of the present disclosure.

FIG. 6 is a cross-sectional view of a mounting portion showing the airbag module in a stored state according to an embodiment of the present disclosure.

FIG. 7 is an example view showing a smartphone mounted on a mounting portion of a housing according to an embodiment of the present disclosure.

FIG. 8 is an example view showing a wireless charging transmitter provided inside a housing according to an embodiment of the present disclosure.

FIG. 9 is an example view showing an airbag module equipped with a wireless charging receiver according to an embodiment of the present disclosure.

FIG. 10 is an example view showing a removable battery being detached from a housing according to an embodiment of the present disclosure.

FIG. 11 is a cross-sectional view showing a removable battery snap-fitted to a battery compartment according to an embodiment of the present disclosure.

FIG. 12 is an example view showing a removable battery being fixed to a battery compartment by a battery lock according to an embodiment of the present disclosure.

FIG. 13 is a bottom perspective view showing a state in which a removable battery is fixed by a battery lock according to an embodiment of the present disclosure.

FIG. 14 is a bottom perspective view showing a housing with a removable battery separated according to an embodiment of the present disclosure.

FIG. 15 is an example view showing a dock provided on the upper part of a housing according to an embodiment of the present disclosure.

FIG. 16 is a side cross-sectional view of a housing provided with a dock at the upper part according to an embodiment of the present disclosure.

FIG. 17 is an example view showing a speaker provided on the front of the housing according to an embodiment of the present disclosure.

FIG. 18 is an example view showing the internal structure of a housing equipped with a speaker according to an embodiment of the present disclosure.

FIG. 19 is a side cross-sectional view of a housing equipped with a speaker according to an embodiment of the present disclosure.

FIG. 20 is an example view showing a light beam provided on the bottom of the housing according to an embodiment of the present disclosure.

FIG. 21 is an example view showing the internal structure of a housing equipped with a light beam according to an embodiment of the present disclosure.

FIG. 22 is an example view of a light beam according to an embodiment of the present disclosure.

FIG. 23 is an example view showing a light beam capable of outputting a warning signal based on distance detection according to an embodiment of the present disclosure.

FIG. 24 is an example view showing how distance detection is performed according to an embodiment of the present disclosure.

FIG. 25 is an example view showing another embodiment of a light beam provided on the bottom of a housing according to an embodiment of the present disclosure.

FIGS. 26A to 26F are example views, each showing an image implemented by another embodiment of a light beam according to an embodiment of the present disclosure.

FIG. 27 is an example view showing a stabilizer provided in the lighting of the housing according to an embodiment of the present disclosure.

FIG. 28 is a side cross-sectional view of a stabilizer provided in the lighting of the housing according to an embodiment of the present disclosure.

FIG. 29 is an example view of a stabilizer equipped with a coil spring according to an embodiment of the present disclosure.

FIG. 30 is an example view of a stabilizer equipped with a leaf spring according to an embodiment of the present disclosure.

### DETAILED DESCRIPTION OF ILLUSTRATIVE EMBODIMENTS

Hereinafter, example embodiments disclosed in the present specification will be described in detail with reference to the attached drawings. Identical or similar components can be assigned same reference numbers regardless of reference numerals, and duplicate descriptions thereof can be omitted. The suffixes “module” and “part” for components used in the following description can be given or used interchangeably for the ease of preparing the specification, and do not necessarily have distinct meanings or roles in themselves. Additionally, in describing the example embodiments disclosed in this specification, if it can be determined that detailed descriptions of related known technologies may obscure the gist of the embodiments disclosed in this specification, accordingly the detailed descriptions of such can be omitted. In addition, it can be understood that the attached drawings are for understanding of the example embodiments disclosed in this specification, and technical ideas disclosed in this specification is not necessarily limited by the attached drawings, and all changes, equivalents, and substitutes included in the spirit and technical scopes of the present disclosure can be included.

Terms including ordinal numbers such as “first” and “second” may be used to explain various components, but the components are not necessarily limited by such terms. For example, such terms can be used merely for the purpose of distinguishing one component from another.

When a component is referred to as “connected” or “linked” to another component, it may be directly connected to or linked to that another component, but it can also be understood that there may be further another component therebetween. On the other hand, when it is mentioned that a component is “directly linked” or “directly connected” to another component, it can be understood that there are no other components in between. In addition, a singular term in the present disclosure can include a plural term unless it is contextually clearly a singular form.

In the present disclosure, terms such as “include” or “have” are to specify that there are features, numbers, steps, operations, components or parts described in the present disclosure, or combinations thereof, and it can be understood that the presence or the possibility of addition of numbers, steps, operations, components, part, or combinations thereof are not excluded in advance.

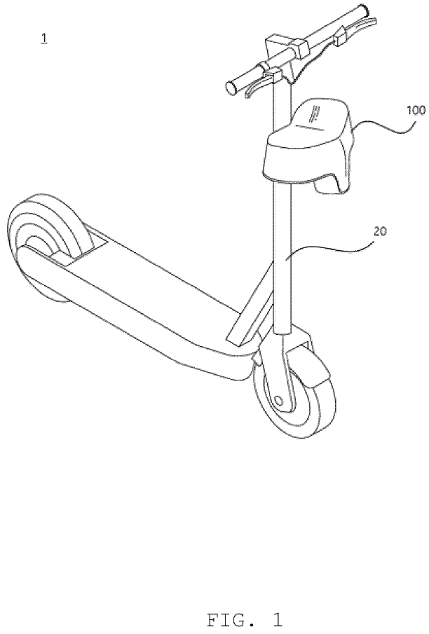

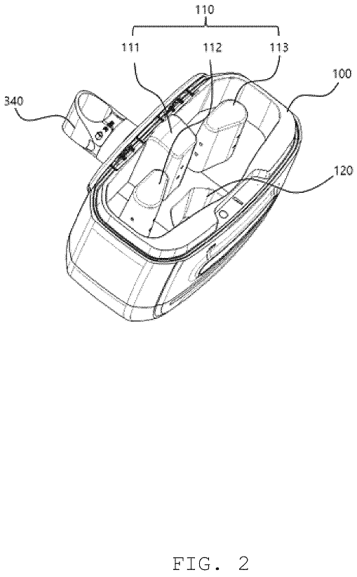

Hereinafter, example embodiments of the present disclosure will be described in detail with reference to the drawings. FIG. 1 is an example view of an electric kickboard to which a safety device storage kit for a personal mobility apparatus according to an embodiment of the present disclosure is applied. FIG. 2 is an internal perspective view of a housing according to an embodiment of the present disclosure.

Referring to FIGS. 1-5, a safety device storage kit for a personal mobility apparatus according to some embodiments of the present disclosure may include a housing 100 in which a convenience device module is stored, a cover 200 configured to cover the upper part of the housing to prevent the inflow of foreign substances, and a steering axis fastening part including a fixing bracket 340 to couple one surface of the housing to the steering axis of the personal mobility apparatus.

The convenience device module may include safety devices including an airbag module that may be worn by the user, or various devices for user convenience. In an embodiment, the convenience device module can be an airbag module, but an embodiment is not necessarily limited thereto.

As shown in FIGS. 1-7, the safety device storage kit of the personal mobility apparatus can be fixed in a steering axis 20 of a personal mobility apparatus 1, and an airbag module 10, which can be a personal safety device, can be stored inside the safety device storage kit to prevent contamination by external contaminants as well as theft or loss.

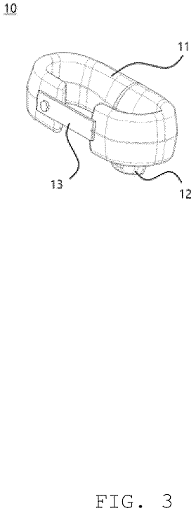

As shown in FIG. 3, the airbag module 10 may include an airbag helmet 11 configured to surround a user's neck when worn, a sensor that detects an external impact applied to the user through the personal mobility apparatus, a control unit that determines whether the airbag is deployed based on the sensing result of the sensor, and an activation device 13 that manipulates whether to switch the sensor and the control unit to an activated state or maintain a deactivated state.

The airbag module 10 can house the sensor and the control unit, can be equipped with a battery that supplies power for the operation of the sensor and the control unit, and may further include a charging module 12 that receives power for charging the battery while stored in the housing.

As shown in FIG. 2, the housing 100 may include an inner wall portion 110 spaced apart to support the inner peripheral surface of the airbag module, and a mounting portion 120 that can be concavely formed at the bottom of the housing and into which the charging module provided in the airbag module can be inserted.

The inner wall portion 110 may include a blocking wall 111 that can protrude from the center of one side wall of the housing toward the inner space so that the inner peripheral surface that comes in contact with the wearer's body may be housed in a state spaced apart from each other to perform sterilization while keeping the airbag module in a deactivated state, a first inner wall 112 that can be located at a distance from one side of the blocking wall and protrude from the bottom of the housing toward the upper space, and a second inner wall 113 that can be located at a distance from the other side of the blocking wall and protrude from the bottom of the housing toward the upper space.

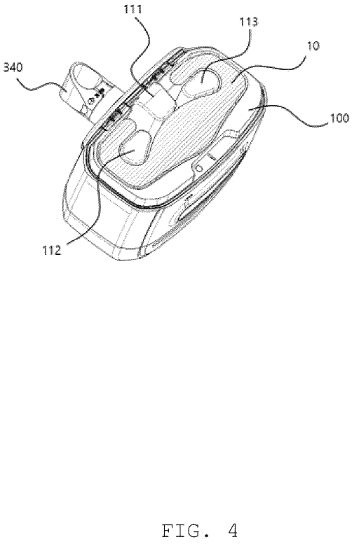

As shown in FIG. 4, the blocking wall 111 can be inserted between the open spaces of the airbag module, and the first inner wall 112 and the second inner wall 113 may be housed in the housing as if docked in a state that the first inner wall 112 and the second inner wall 113 can be respectively inserted into the inner peripheral surfaces of both ends of the airbag module.

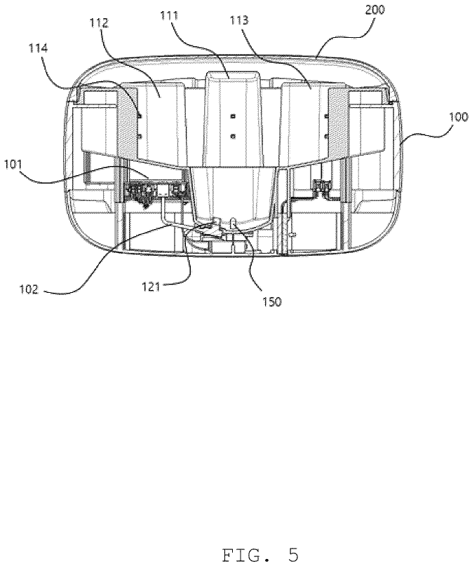

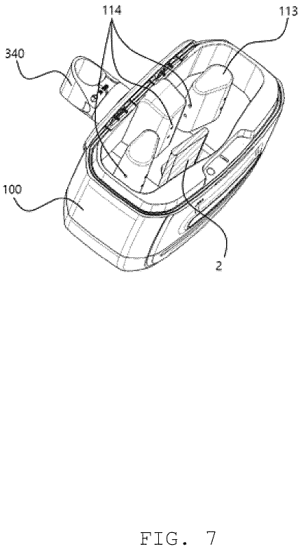

As shown in FIGS. 5 and 7, the front side of the blocking wall 111 and the circumference of the first inner wall 112 and the second inner wall 113 can be provided with a UVC LED 114 for disinfecting the inner peripheral surface of the airbag module.

The UVC LED 114 can be a light emitting diode that performs sterilization and disinfection by damaging DNA and RNA molecule structures of bacteria by generating ultraviolet rays with a wavelength of 200 to 280 nm. Accordingly, the airbag module may maintain a clean condition because the UVC LED may disinfect the area that was in contact with the body while using a personal mobility apparatus such as an electric kickboard while the airbag module is stored inside the housing after use.

As shown in FIGS. 2-5, the mounting portion 120 can be a space into which the charging module 12 provided in the airbag module can be inserted, and can be formed concavely at the bottom of the housing 100, and a charging terminal 121 to which the charging module can be electrically connected can be provided on the bottom of the mounting portion 120 to charge the battery provided in the airbag module.

The mounting portion 120 may be configured in various shapes considering the shape and location of the charging terminal provided in the charging module. The mounting portion 120 is shown to be located in front of the blocking wall 111, but it is not necessarily limited to this and may be formed in various ways depending on the shape and position of the charging module stored in the housing.

The inner space of the housing 100 may be utilized as a storage space while the airbag module is taken out and worn by the user to operate the personal mobility apparatus.

In particular, because the mounting portion 120 can be provided with a charging terminal 121, as shown in FIG. 7, the charging terminal of a smart terminal such as a smartphone or tablet PC may be electrically connected to the charging terminal 121 of the mounting portion 120 to allow charging.

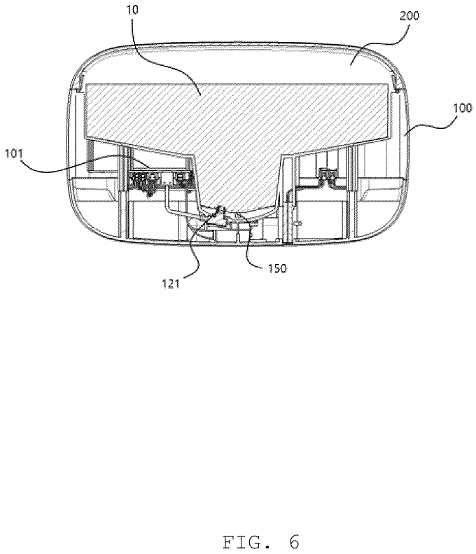

A pressure sensor 150, which can be pressed by the load of the airbag module disposed at the top, and return to its original state and detect whether the object is to be disposed when the airbag module is removed, may be further included at the bottom of the mounting portion 120 as illustrated in FIGS. 5 and 6.

Not only the pressure sensor 150 but also a charging terminal 121 for charging the airbag module or smart terminal as a power supply may be provided on the bottom of the mounting portion 120.

The pressure sensor 150 may measure a change in stroke value caused by being pressed by the weight or shape of an object disposed in the mounting portion, and transmit the information on the change to the control unit 101 provided in the housing.

Accordingly, the controller or control unit 101 can determine whether the airbag module is properly stored based on the change of the stroke value transmitted in the pressure sensor, thereby preventing an error in which the personal mobile device is returned while another object is stored.

The control unit 101 may determine whether the airbag module is stored in its normal position, based on the appropriateness of the change in stroke value received from the pressure sensor (for example, when the stroke value before storing the airbag module is 0 mm, and the stroke value after storing the airbag module is 5 mm).

When it is determined that the airbag module is stored in a normal position, the control unit 101 may determine that an electrical connection is made between the battery of the charging module provided in the airbag module and the charging terminal provided in the housing, and perform charging.

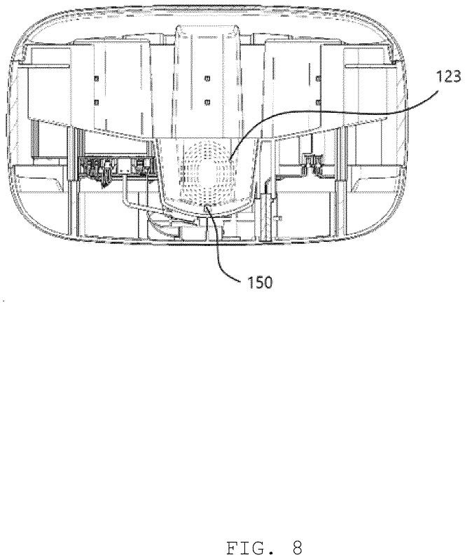

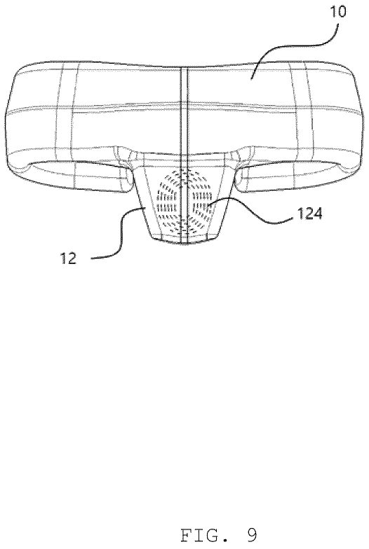

The charging terminal may be configured as a contact or wired charging terminal as shown in FIGS. 5 and 6, and may also be configured as a wireless charging terminal as shown in FIGS. 8 and 9.

As shown in FIGS. 5 and 6, when it is determined that the airbag module is stored at the normal position of the mounting portion by the pressure sensor, the control unit 101 may determine that an electrical connection is made between the battery terminal of the charging module provided in the airbag module and the charging terminal 121, and perform charging.

The charging terminal 121 and the battery terminal of the charging module may be composed of various wired charging means that can be contacted or combined for wired charging.

The charging components for charging the battery provided in the charging module may be configured as a wireless charging device, as shown in FIGS. 8 and 9. The mounting portion 120 can be provided with a first wireless charging transmitter 123 formed of a coil that can wirelessly supplies power as shown in FIG. 8, and the charging module of the airbag module may be provided with a first wireless charging receiver 124 formed of a coil that can charge the battery by receiving power supplied from the wireless charging transmitter, as shown in FIG. 9.

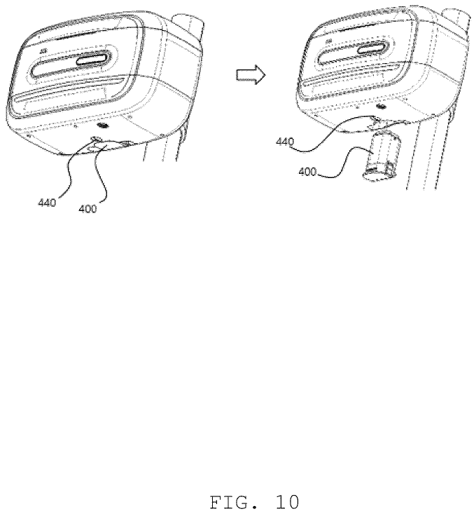

Referring to FIGS. 10-14, the housing 100 may include a removable battery 400 that can supply power for charging the airbag module through the charging module, and a battery compartment 410 can be formed on the bottom of the housing to enable electrical connection to the charging module, as a space where the removable battery can be mounted. The left side of FIG. 10 shows a state in which the removable battery 400 is mounted in the battery compartment 410, and the right side of FIG. 10 shows a state in which the removable battery is separated.

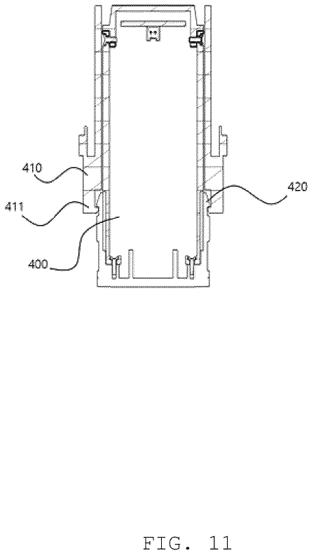

As shown in FIG. 11, the removable battery 400 can have a battery hook 420 formed on the outer side for snap-fit coupling, and a battery locking protrusion 411 on that the battery hook can be hung may be formed in the battery compartment 410.

Accordingly, just by pushing the removable battery into the battery compartment, the battery hook may be easily coupled to the battery locking protrusion.

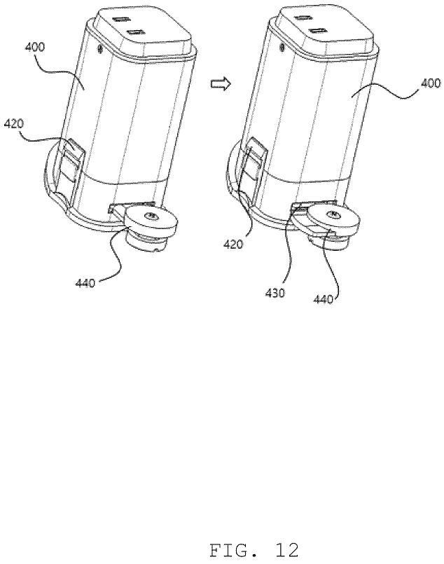

Referring to FIGS. 12-14, the removable battery 400 can be provided with a battery groove 430 that can be concavely formed toward the inside for battery locking, and the battery compartment may be provided with a battery locking device 440 that can be inserted into or removed from the battery groove by rotation.

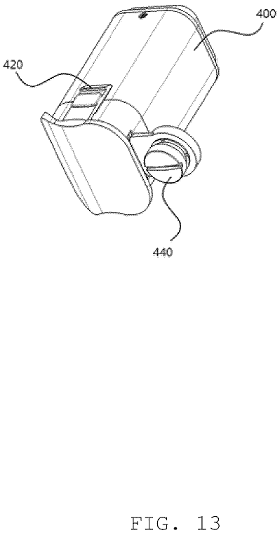

As shown on the left side of FIG. 12 and FIG. 13, the battery locking device 440 can be inserted into the battery groove 430 and kept in a locked state while the removable battery is mounted in the battery compartment, thereby preventing the removable battery from being separated by unexpected external shock.

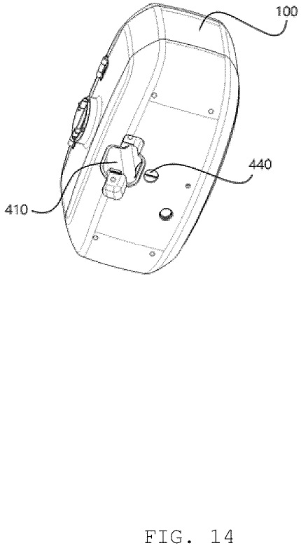

As shown on the right side of FIG. 12 and in FIG. 14, to release the removable battery, the battery locking device 440 may be rotated to be removed from the battery groove 430, and the battery locking device 440 may then be separated from the battery compartment by manipulating the battery hook 420.

The battery locking device can be allowed to be rotated by inserting a thin object such as a key or a special tool into a concave manipulation unit to prevent easy rotation by an unexpected external force.

The removable battery 400 may be charged by receiving power through a power line connected to a power source such as a separate external battery while mounted in the battery compartment 410, thereby improving convenience of use. The bottom of the housing may be provided with a power terminal to which a power line for supplying power to the removable battery can be connected.

The cover 200 can be a plate that covers the upper part of the housing to prevent theft of the airbag housing, and may be rotatably coupled to the housing 100 by a hinge 210.

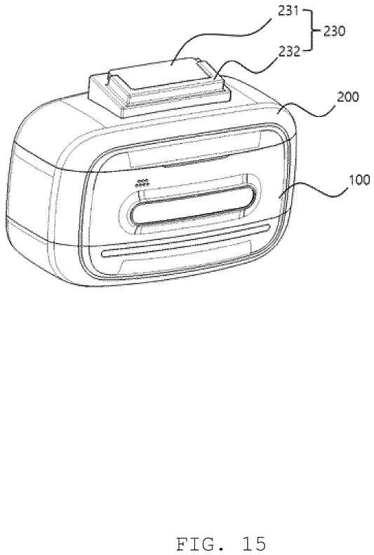

As shown in FIG. 15, the upper part of the cover may be further provided with a smartphone holder 230 that can hold the smartphone while using a personal mobility apparatus.

The smartphone mounting portion 230 may include a mounting plate 231 installed high on one side and low on the other side to place the smartphone at an angle so that it faces the user's field of view, and a grip portion 232 bent to surround both sides and a lower surface of the mounting plate.

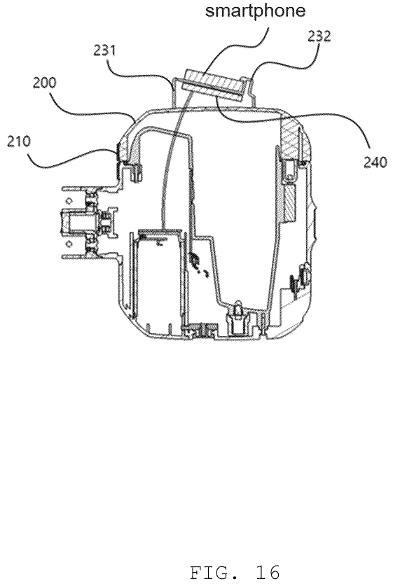

As shown in FIG. 16, the smartphone placed on the holder plate 231 can be coupled as if inserted into the grip portion 232, thereby preventing the smartphone from being separated from the mounting portion even while the personal mobility apparatus is running.

Further, a second wireless charging transmitter 240 for charging the smartphone using power supplied through a wire or the like from a removable battery can be provided in the housing and may be further provided on the bottom of the mounting plate 231 for wireless charging of the smartphone placed on the mounting plate.

While using a personal mobility apparatus, the user can view the screen while charging the smartphone and use it for purposes such as navigation.

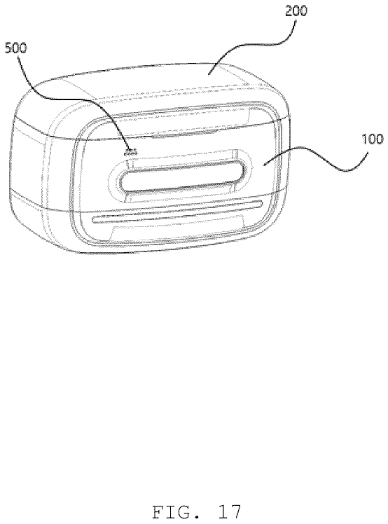

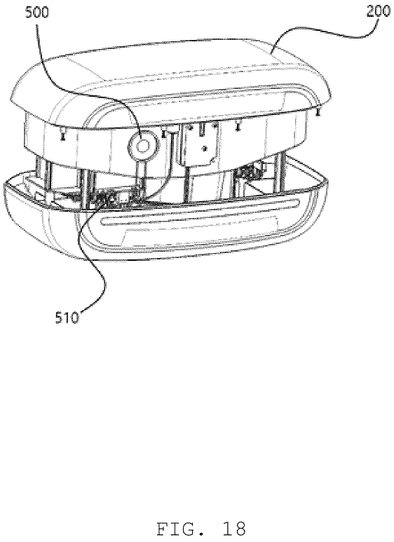

The housing 100 may be further equipped with a speaker 500 that outputs audio, as shown in FIGS. 17 to 19. A Bluetooth module 510 that enables wireless communication with the smartphone may be further provided to enable output of music played on the smartphone, etc. of a user using a personal mobility apparatus through the speaker.

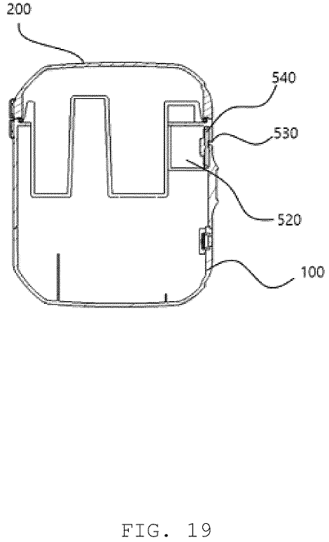

As shown in FIG. 19, a speaker frame 520 can be installed inside the housing to assist the sound of the speaker, a speaker hole 530 can be installed inside the speaker frame 520 and penetrate through the outer peripheral surface of the housing, and a waterproof film 540 can block moisture flowing in through the speaker hole 530.

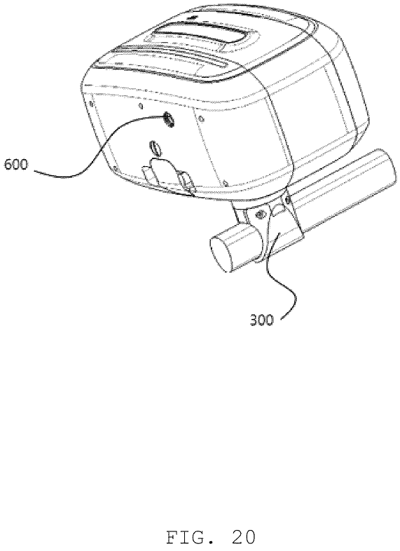

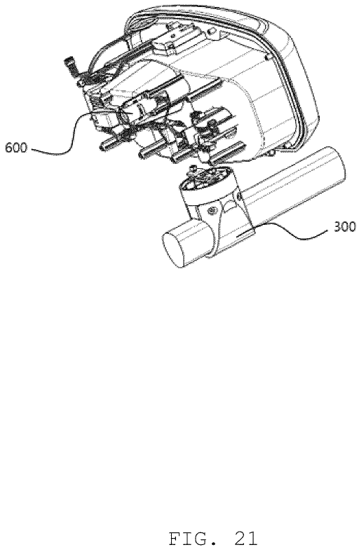

As shown in FIGS. 20 and 21, the housing 100 may further include a light beam generator 600 that can generate a beam of a certain image and project it on the ground to notify surroundings of the approach of the personal mobility apparatus.

In other words, by generating and projecting a beam of a certain image onto the ground in front of the personal mobility apparatus, visibility to surrounding people regarding the approach and location of the personal mobility apparatus may be increased.

The light beam generator 600 may be installed on the bottom of the housing at a certain angle to face forward, as shown in FIGS. 20 and 21. Accordingly, it can be possible to prevent the light beam, which is irradiated from the light beam generator 600, from being directly irradiated into the field of view of surrounding people.

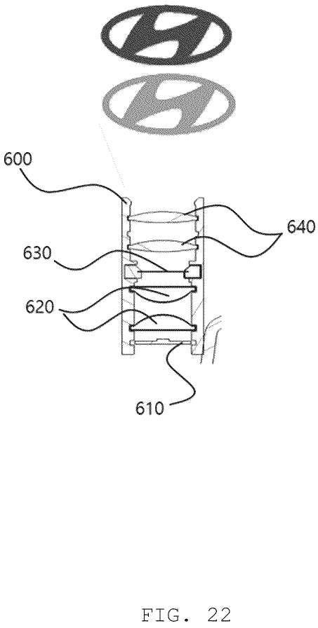

As shown in FIG. 22, the light beam generator 600 may include an LED 610 that generates light, a first lens 620 that transmits light generated by the LED to the front, an image film 630 that provides an image to be projected using light passing through the first lens, and a second lens 640 that projects the light passing through the image film onto the ground outside the housing. The second lens 640 becomes the outermost lens for projecting an image onto an area outside the housing.

In this way, the image generated by the light beam generator 600 may project a certain image as shown in FIG. 22 onto the ground in front of the personal mobility apparatus, thereby making surrounding people aware of the approach of the personal mobility apparatus. The projected image may be configured in various ways depending on the content implemented on the image film.

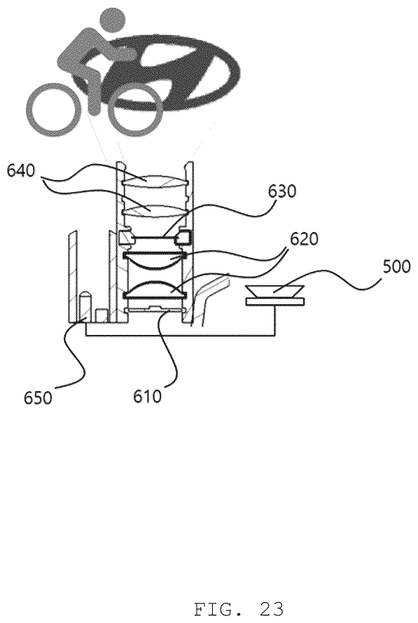

The light beam generator 600 may further include a distance detector 650 that measures the distance between the personal mobility apparatus and a pedestrian or obstacle in front of the personal mobility apparatus, as shown in FIG. 23.

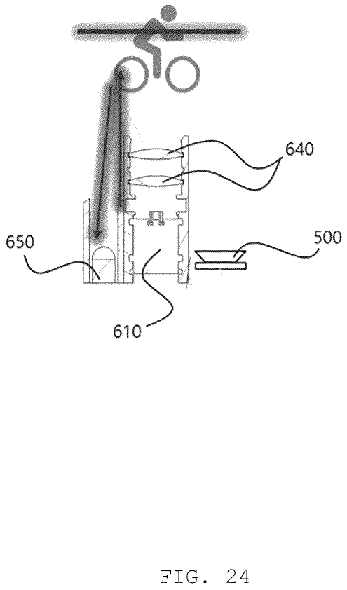

The distance detector 650 may be composed of an infrared transceiver or an ultrasonic sensor installed on one side of the light beam generator. As shown in FIG. 24, when a pedestrian passes a place where an image is projected, the distance detector 650 may measure the distance to the pedestrian using information received when the infrared rays emitted from the infrared transmitter are reflected to the pedestrian.

When the distance to the pedestrian is less than a preset or certain distance according to the information measured by the distance detector 650, there can be a risk of collision with the pedestrian, so a certain warning sound may be transmitted through the speaker 500. This warning sound can enable not only users using the personal mobility apparatus but also pedestrians to recognize the approach of the personal mobility apparatus and take prompt action. In FIGS. 23 and 24, a pedestrian is shown in the shape of a person riding a bicycle.

Additionally, the light beam generator 600 may change the color of the image projected on the ground, based on a preset or selected reference running speed of the personal mobility apparatus.

As shown in FIG. 22, when the speed of the personal mobility apparatus is slower than the reference speed, the color of the image projected on the ground may be set to the first color (e.g., green) and closer, and when the speed of the image projected on the ground is set to be faster than the reference speed, the color of the image projected on the ground may be changed to a second color (e.g., red) and further ahead to urge the attention of pedestrians.

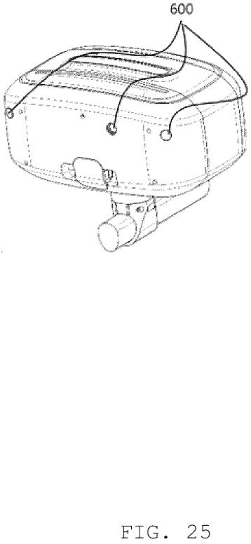

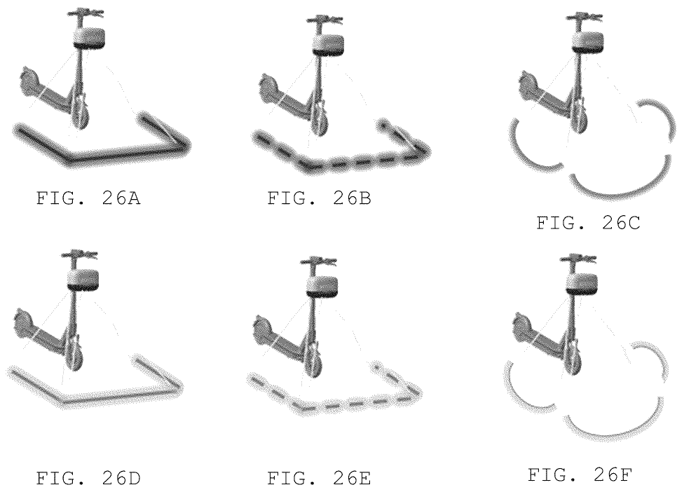

As shown in FIGS. 25 and 26A-26F, a plurality of light beam generators 600 may be provided in the housing to enable simultaneous projection of multiple images on the ground.

As shown in FIG. 25, a second light beam generator and a third light beam generator may be further provided on both sides of the first light beam generator provided near the center of the bottom of the housing. The first to third light beam generators may each generate preset or selected images and project the combined image onto the ground as shown in FIGS. 26A-26F.

FIGS. 26A to 26C show that a red image can be projected when the speed of the personal mobility apparatus exceeds a preset or selected reference speed, and FIGS. 26D to 26F show that a green image can be projected when the speed of the personal mobility apparatus does not exceed the preset or selected reference speed.

The images projected from the first to third light beam generators may be composed of straight lines as shown in FIGS. 26A and 26D, composed of dotted lines as shown in FIGS. 26B and 26E, or composed of curves as shown in FIGS. 26C and 26F, for example.

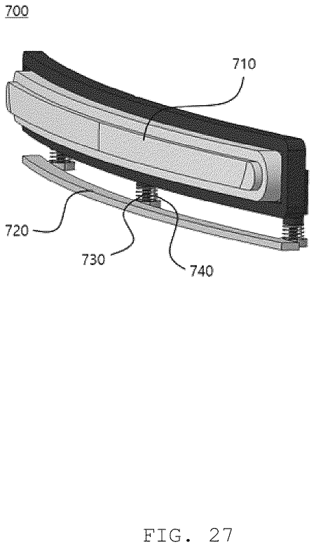

As shown in FIG. 27, the housing 100 may further include a lighting unit 710 and a stabilizer 700. The stabilizer 700 can attenuate vibrations transmitted to the lighting unit 710 while the personal mobility apparatus is running to minimize vibration of the lighting provided from the lighting unit 710.

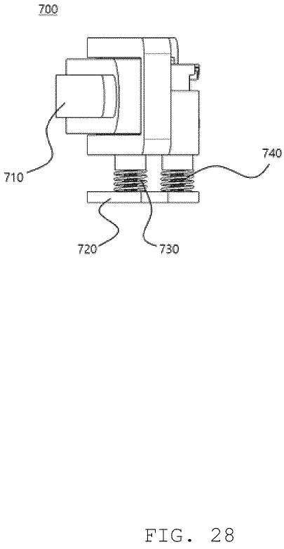

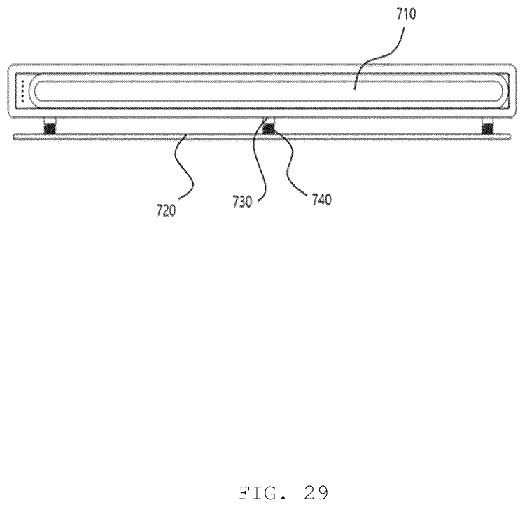

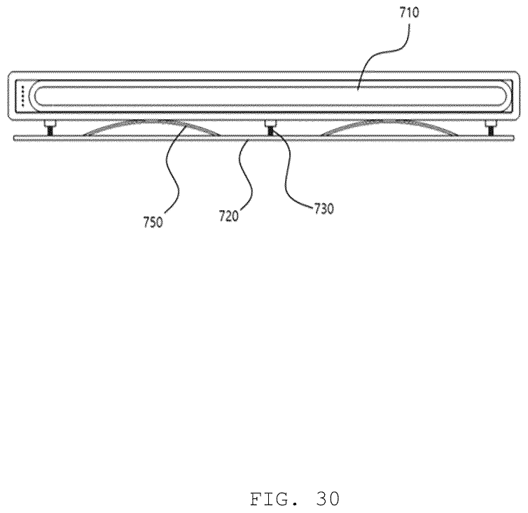

As shown in FIGS. 27 to 29, the stabilizer 700 may include a lower fixing member 720 and at least one support bar 730. The lower fixing member 720 can include a lower fixing surface that is attached to the personal mobility apparatus when the housing 100 is attached thereto, and the at least one support bar 730 can separate the bottom of the lighting unit 710 from the lower fixing member 720. The at least one support bar 730 can be compressible so as to guide movement of the lower fixing member 720 toward the lighting unit 710. In an example embodiment, the at least one support bar 730 may include a telescopic structure that is compressible. Also, the stabilizer 700 may further include a spring mounted between the lighting unit 710 and the lower fixing member 720 to attenuate the vibration. The spring may be a coil spring 740 as shown in FIG. 28 and FIG. 29, or a plate/leaf spring 750 as shown in FIG. 30.

Accordingly, vibration or shock transmitted from the ground while the personal mobility apparatus is running may be attenuated by the coil spring 740 before being transmitted to the lighting unit 710, thereby minimizing the vibration of the lighting unit. As such, light projected from the lighting unit may be stabilized.

As shown in FIG. 30, the stabilizer 700 may include, instead of a coil spring 740, a leaf spring 750 that is located between the lighting unit 710 and the lower fixing member 720. In this case, vibration or shock transmitted from the ground can be attenuated by the leaf spring 750 and then transmitted to the lighting unit 710, thereby contributing to lighting stabilization while minimizing vibration of the lighting unit.

The above detailed description should not be necessarily construed as restrictive in any respect and can be considered illustrative. The scopes of the present disclosure can be determined by reasonable interpretation of the appended claims, and all changes within equivalent scopes of the present disclosure can be included in the scopes of the present disclosure.

What is claimed is:

1. A storage kit for a personal mobility apparatus, the storage kit comprising:
a housing configured to accommodate a convenience device module, the housing including a power supply device coupled to the convenience device module;
a cover configured to cover an upper part of the housing; and
a fastening part configured to couple the housing to the personal mobility apparatus.

2. The storage kit of claim 1, wherein the housing includes a light beam generator configured to project image beam on a ground, and wherein the light beam generator includes:
a LED configured to generate light;
a first lens configured to transmit the light;
an image film configured to provide an image to be projected using light passing through the first lens; and
a second lens, which is an outermost lens configured to project the light passing through the image film onto the ground outside the housing.

3. The storage kit of claim 2, wherein the light beam generator further includes a distance detector configured to measure a distance between the personal mobility apparatus and a pedestrian or obstacle in front of the personal mobility apparatus.

4. The storage kit of claim 3, wherein the distance detector includes an infrared transceiver or ultrasonic sensor installed in the light beam generator.

5. The storage kit of claim 3, wherein the light beam generator includes a speaker, and wherein the light beam generator is configured to transmit a warning sound through the speaker in response to the distance to the pedestrian being less than a first distance.

6. The storage kit of claim 2, wherein the light beam generator is configured to control a color of the image projected on the ground to be changed from a first color to a second color in response to a running speed of the personal mobility apparatus exceeds a reference speed.

7. The storage kit of claim 6, wherein the light beam generator is configured to display the color of the image as the first color in response to the running speed being smaller than the reference speed, and display the color of the image as the second color in response to the running speed being greater than the reference speed.

8. The storage kit of claim 2, wherein the housing includes a plurality of light beam generators, each of the light beam generators being capable of independently displaying an image, thereby providing simultaneous projection of a plurality of images on the ground.

9. The storage kit of claim 1, wherein the convenience device module includes an airbag module wearable by a user;
wherein the housing includes:
an inner wall configured to support an inner peripheral surface of the airbag module, and
a mounting portion having a concave shape at a bottom of the housing configured for a charging module of the airbag module to be inserted therein; and
wherein the inner wall includes an ultraviolet-spectrum light-emitting diode configured for disinfecting the inner peripheral surface of the airbag module.

10. The storage kit of claim 9, wherein the power supply device comprises a charging terminal at the mounting portion and configured to supply power for charging a battery provided in the charging module of the airbag module or a smart terminal, and
wherein the power supply device further comprises a first wireless charging transmitter installed at the mounting portion and comprising a coil capable of wirelessly supplying power to a first wireless charging receiver of the charging module of the airbag module.

11. The storage kit of claim 1, wherein the cover comprises a smartphone mounting portion configured for mounting a smartphone, and
wherein the smartphone mounting portion includes:
a mounting plate slanted and configured to place the smartphone at an angle thereon; and
a grip portion configured to surround lateral sides and a lower surface of the mounting plate.

12. The storage kit of claim 11, wherein a second wireless charging transmitter for wirelessly charging the smartphone placed on the mounting plate is at a bottom surface of the mounting plate.

13. The storage kit of claim 11, wherein the housing includes:
a wireless communication module configured to enable wireless communication with the smartphone; and
a speaker configured to output audio played by the smartphone.

14. The storage kit of claim 1, wherein the housing includes a lighting unit and a stabilizer, wherein the stabilizer is configured to attenuate vibration transmitted to the lighting unit while the personal mobility apparatus is running.

15. The storage kit of claim 14, wherein the stabilizer includes:
a lower fixing member comprising a lower fixing surface attached to the personal mobility apparatus;
at least one support bar mounted between the lighting unit and the lower fixing member and configured to be compressible to guide movement of the lower fixing member toward the lighting unit; and
a spring mounted between the lighting unit and the lower fixing member.

16. The storage kit of claim 15, wherein the spring includes:
a coil spring configured to surround the at least one support bar; or
a plate spring mounted between the lighting unit and the lower fixing member.

17. A storage kit for a personal mobility apparatus, the storage kit comprising:
a housing configured to accommodate a user-wearable safety device therein; and
a fastening part configured to couple the housing to the personal mobility apparatus,
wherein the housing includes a power supply device configured to supply power to the user-wearable safety device,
wherein the housing includes a light beam generator device configured to project image beam on a ground, and wherein the light beam generator device includes:
a LED configured to generate light,
a first lens configured to transmit the light,
an image film configured to provide an image to be projected using light passing through the first lens,
a second lens, which is an outermost lens configured to project the light passing through the image film onto the ground outside the housing,
a distance detector configured to measure a distance between the personal mobility apparatus and a pedestrian or obstacle in front of the personal mobility apparatus, wherein the distance detector includes an infrared transceiver or ultrasonic sensor installed in the light beam generator device, and
a speaker, wherein the light beam generator device is configured to transmit a warning sound through the speaker in response to the distance to the pedestrian being less than a first distance,
wherein the light beam generator device is configured to control a color of the image projected on the ground to be changed from a first color to a second color in response to a running speed of the personal mobility apparatus exceeds a reference speed, and wherein the light beam generator device is configured to display the color of the image as the first color in response to the running speed being smaller than the reference speed, and display the color of the image as the second color in response to the running speed being greater than the reference speed.

18. The storage kit of claim 17, wherein the housing includes a plurality of light beam generator devices, each of the light beam generator devices being capable of independently displaying an image, thereby providing simultaneous projection of a plurality of images on the ground.

19. The storage kit of claim 17, wherein the housing includes a lighting unit and a stabilizer, wherein the stabilizer is configured to attenuate vibration transmitted to the lighting unit while the personal mobility apparatus is running.

20. A storage kit for a personal mobility apparatus, the storage kit comprising:
an airbag module wearable by a user;
a housing configured to accommodate the airbag module therein; and
a fastening part configured to couple the housing to the personal mobility apparatus, wherein the housing includes a power supply device configured to supply power to the airbag module.
